(19) United States
(12) Patent Application Publication
(10) Pub. No.: US 2026/0346009 A1
(43) Pub. Date: Nov. 5, 2026

(54) NEUROSTIMULATION DEVICE AND SYSTEM FOR TRANSCUTANEOUS AURICULAR BRANCH VAGUS NERVE STIMULATION

(71) Applicant: Wai Kin Daniel KO, Hong Kong (CN)

(72) Inventor: Wai Kin Daniel KO, Hong Kong (CN)

(21) Appl. No.: 19/115,460

(22) PCT Filed: Oct. 25, 2023
(86) PCT No.: PCT/CN2023/126503

§ 371 Date: Mar. 26, 2025

Related U.S. Application Data
(60) Provisional application No. 63/420,809, filed on Oct. 31, 2022.

(51) Int. Cl. A61N 1/36; A61N 1/04

(52) U.S. Cl. CPC A61N 1/36036; A61N 1/0456; A61N 1/0472; A61N 1/36031

(57) ABSTRACT

The present invention refers to a neurostimulation device (1) that is wearable by a user to stimulate the Auricular Branch of Vagus Nerve (ABVN) of the user's ear. The device (1) is wireless and comprises of: at least 1 electrode (2) designed to be located in the cymba conchae for stimulation of the Ramus Auricularis Nervi Vagi (RANV) when an electrical voltage difference is applied; and an infinity shape holding portion (3) designed to hold the device at the user's ear. The invention further refers to a neurostimulation system that comprises of the wireless device (1) and a controller unit (8), with the neurostimulation system having at least one detection means configured to detect one or more parameter values, wherein feedback data are sent to a dedicated cloud platform, and with the system suitable to set parameters of the neurostimulation delivered by the device (1) based on feedback parameter values.

### FIELD OF INVENTION

The present invention relates to an auricular neurostimulation device. The invention further refers to an auricular neurostimulation system that comprises of the neurostimulation device that can be adapted, personalized and applied in synchronization with combined neurostimulation device (electrode assemblies), according to the user's requirements, as such having higher efficiency and stability for delivering stimulation. The invention further relates to a method for operation and deliverance of such a neurostimulation system.

### BACKGROUND

The Vagus Nerve (VN), the 10^{th} cranial nerve, is the longest cranial nerve and connects the brainstem to the body from the medulla to the colon and predominately innervates thoracic and abdominal organs, playing a key role in enabling the brain to regulate and monitor essential bodily functions and homeostasis. The VN is a major component of parasympathetic regulation of the Autonomic Nervous System (ANS) and forms a neural circuit that regulates key physiology including heart rate, gastrointestinal motility, and secretion such as pancreatic secretion and glucose production.

The VN constitutes a bilateral neural structure extending from the brainstem through the neck, abdomen, and main organs with pivotal roles in both efferent and afferent functions. The VN encompasses approximately 80-90% of afferent nerves. These afferent pathways serve to transmit sensory information of the internal state of major organs to the Central Nervous System (CNS). This sensory feedback is integral to the function of the neuro-endocrine-immune axis that actively contributes to the maintenance of homeostasis. These afferent pathways also engage with key components of physiological control, including the hypothalamic-pituitary-adrenal axis and the ANS.

The VN has extensive reciprocal connections that integrate and respond to feedback signals, with connections that extend to vital systems including cardiovascular, respiratory, gastrointestinal, autonomic, immune and endocrine functions. The VN is mainly composed of cholinergic fibres, but also mediates non-cholinergic and non-adrenergic transmitters. The VN enters the CNS via the Nucleus Tractus Solitarius (NTS) that extends through afferent projections to the parabrachial complex (PB). The PB forms connections with the Locus Coeruleus (LC) and Raphe Nuclei, where further ascending projections reach higher brain regions, regulating key brain processes.

With key effects on various physiological systems for homeostasis, the VN has been targeted for modulating organ functions for therapeutic applications. Invasive cervical Vagus Nerve Stimulation (VNS) was approved by the US FDA in 1997 for treatment-resistant epilepsy and in 2005 for drug-resistant depression. VNS is a recognized clinical technique that involves direct administration of electrical impulses to the VN that is accomplished using an implantable device beneath the skin and commonly a spiral electrode that is surgically attached to the left cervical vagus nerve in the neck region.

The medical devices for VNS were first developed by Cyberonics Inc (now known as Livanova Inc). Other implantable medical devices for VNS have been developed that include:
Vivistim by MicroTransponder for stroke
SetPoint Medical for Rheumatoid Arthritis and inflammatory disorders
CardioFit by BioControl Medical for congestive heart failure
G112 by Pins Medical for pediatric drug resistant epilepsy Even with clinical adoption of prominent VNS devices, there are limitations for wider adoption, which includes surgical risks and associated adverse effects. The VNS with implantable devices is an irreversible procedure that presents risks of infection (with approximately 3% incidence) and can cause potential nerve damage. Voice alterations i.e. hoarseness during stimulation or cough pressure are risks tied to laryngeal recurrent nerve impact (with approximately 5% incidence). Other side effects of VNS treatment include dyspnea, local pain and abnormal muscle contractions and are caused by bi-directional stimulation of VN fibres.

To address the challenges associated with invasive VNS devices, non-invasive approaches like Transcutaneous Vagus Nerve Stimulation (tVNS) have gained prominence. This method involves delivering electrical stimulation to the skin overlying the VN, offering neuromodulation without surgical intervention. tVNS commonly utilizes a portable device applied to the neck or ear. The device's electrical impulses activate nerve fibres under the skin, in turn stimulating the VN and transmitting signals to the brainstem and higher brain regions. tVNS provides an effective low-risk alternative therapy with significant potential across various clinical applications.

tVNS can be achieved via activation of the Auricular Branch of Vagus Nerve (ABVN) and was first described by Ventureyra et al. (2000). The ABVN nerve extends to the pinna of the ear and can be electrically depolarized with minimal invasiveness. tVNS at the cymba conchae induces activation of the primary termination sites of the main vagal sensory pathways, the spinal trigeminal nucleus and the Nucleus Tractus Solitary (NTS) (Frangos et al., 2015). The neurostimulation leads to activation of brainstem nuclei, such as the Locus Coeruleus (LC) and parabrachialis nucleus, and activation of mid-brain areas including the periaqueductal grey matter, dorsal raphe nucleus, substantia nigra and red nucleus (Frangos et al., 2015, Badran et al., 2017).

tVNS has been used in a range of clinical indications such as depression, anxiety, schizophrenia, autism, epilepsy (Rong, 2014; Stefan et al., 2012; Bauer et al., 2016), migraine (Straube et al., 2015), pre-diabetes (Huang et al., 2014) and post-ischemic stroke rehabilitation recovery (Baig et al., 2019; Redgrave et al., 2018). In addition, it has also been used to for improving memory function (Jacob et al., 2015) and is a potential therapeutic tool for Alzheimer's disease (Kaczmarczyk et al., 2017; Cai et al., 2019) and other neurodegenerative disorders like Parkinson's disease (Ko, 2021).

tVNS medical devices offer portability and user benefit from autonomy, customizable dosing, medication compatibility due to non-drug interactions and therefore lack of systemic side effects and/ or development of treatment resistance. Devices in the state of the art for ABVN stimulation include Nemos® from Cerbomed, Nurosym from Parasym Ltd, Nervana® from Nervana LLC and Net1000® from Auri-Stim Medical, as well as others.

However, tVNS medical devices in prior art commonly target regions that are not exclusively supplied by the ABVN such as the cavity of conchae and tragus (for example Nurosym from Parasym Ltd). It has been shown that the cymba conchae is the only anatomical location exclusively innervated by the ABVN (Peuker and Filler, 2002).

The neurostimulation device Nemos® by Cerbomed stimulates the cymba conchae (EP3100764 (A1) and DE102015007215 (B3)). However, due to the form factor of device electrode and variation of size of user's ears, there is sub-optimal contact of electrodes with the cymba conchae and poor ABVN stimulation that reduces the efficiency of the device. Furthermore, the devices are as a wired design, requiring physical connections for stimulation making the device inconvenient to use due to size and are not suited for continued application with a limited wear time for therapy.

It has been described that electrical stimulation of the ABVN is an emerging therapeutic application with its diverse control of key physiological mechanisms between the brain and body. As such there is high potential of new technology that provides user optimizations, efficiency, personalization and comfort in use.

The object of the invention introduces a wireless wearable neurostimulation device for tVNS stimulation of the nerve ramifications of the cymba conchae for ABVN neurostimulation, with increased efficiency, comfort, and offers personalization via feedback analyses from user data that can be adapted to each individual user's needs.

### SUMMARY OF INVENTION

Considering the aforementioned prior art, the object of the present invention as per a first aspect, a wearable auricular neurostimulation device. This neurostimulation device (electrode assembly) is wireless and is intended for user wear for stimulation of the ABVN of the user's ear. The neurostimulation device (electrode assembly) incorporates at least one electrode positioned at the cymba conchae. The electrodes effectively utilize the entire cymba conchae to stimulate the ABVN when a voltage difference is applied.

The present invention refers to a neurostimulation device (1) that is wearable by a user and is configured to stimulate the Auricular Branch of Vagus Nerve (ABVN) of the user's ear. The invention further refers to a neurostimulation system that comprises of the neurostimulation device (electrode assembly) (1) and controller unit (8). The neurostimulation device (electrode assembly) (1) is wireless and comprises of at least 1 electrode (2) designed to be located in the cymba conchae, where the electrode stimulates the Ramus Auricularis Nervi Vagi (RANV), when an electrical voltage difference is applied. The neurostimulation device (electrode assembly) (1) comprises of at least 1 electrode (2) for generating a stimulating pulse, with the neurostimulation system having at least one detection means that is configured to detect one or more parameter values, and with the system having a controller unit (8) that is suitable to set one or more parameters of the stimulation pulse delivered by the neurostimulation device (electrode assembly) (1) based on parameter value or values. The neurostimulation device (electrode assembly) (1) comprises of at least 1 electrode (2) and an infinity shape ('figure of eight') holding portion (3) that is designed to hold the electrode assembly at the ear of the user.

The invention further refers to a neurostimulation system for Auricular Branch of Vagus Nerve (ABVN) stimulation, having at least one electrode (2) for generating a stimulation pulse, wherein the neurostimulation system has at least one input device for inputting feedback data by the device user, wherein the neurostimulation system has memory in which the feedback data are stored or sent to a dedicated platform in the cloud, and where in the neurostimulation system has a controller unit (8) suitable to set one or more parameters of the stimulation pulse that is delivered wirelessly by the neurostimulation device (electrode assembly) (1), or to provide adopted parameters of the stimulation pulse delivered by the neurostimulation device (electrode assembly) (1) for selection by the user. Further, the success of the treatment delivered by the neurostimulation device (electrode assembly) (1) can take place in a manner specific to the user (personalized) or that the parameters that may influence stimulation, can determine a stimulation that increases the success of the treatment with respect to an unchangeable stimulation pattern i.e. stimulation protocol.

The invention further refers to a neurostimulation system for Auricular Branch of Vagus Nerve (ABVN) stimulation, that can be applied with 2 wireless neurostimulation devices (electrode assemblies) (1) concurrently, both generating a stimulation pulse, wherein the controller unit (8) of the neurostimulation system mediates synchronized or desynchronized stimulation of pulse parameters by the electrode assemblies.

Preferably, the electrodes are made from biocompatible materials (including titanium, nickel titanium, platinum and platinum-iridium, non-toxic metals such as gold) and biocompatible polymers for adaptation to user ear ergonomics for comfort, secure wear that is used for providing accurate positioning and contact to the stimulation area. Further, neurostimulation device (electrode assembly) fitting does not block the user's ear canal i.e. does not impede auditory perceptions during use for carrying out ABVN stimulation.

The neurostimulation device (electrode assembly) is an inventive electrode assembly for nerve stimulation, in particular for ABVN stimulation that includes at least one electrode and an infinity shape (‘figure of eight’) holding portion which is designed to hold the electrode assembly at the ear of the user.

As the activity of the nerve stimulation largely depends on a good contact between the electrode and the tissue to be stimulated, it has proven advantageous in practice when the connecting portion by means of which the electrode is connecting to the holding portion is inclined relative to the connecting portion.

The neurostimulation device (electrode assembly) of the invention includes a conduction sensor that indicates appropriate skin contact of the cymba conchae with the electrodes and therefore good wear integrity.

Preferably, in the auricular neurostimulation device (electrode assembly) of the invention, is used in combination with two wireless stimulation electrodes for each of the user's ears for bilateral ear stimulation, with the stimulation of each ABVN of the user that can be synchronized or unsynchronized.

The auricular neurostimulation device (electrode assembly) of the invention implements stimulation protocols based on waveform of rectangular, biphasic, and symmetric pulse forms.

In one preferred embodiment, the auricular neurostimulation device (electrode assembly) of the invention is configured to coordinate stimulation pulse or pulses with periodically occurring physiological processes, in particular neuro-physiological processes of the user.

Typically, the invention comprises of a smart-phone application that allows the user to interact with the neurostimulator.

According to a second object, the invention relates to an auricular neurostimulation system for ABVN stimulation comprising of the wireless neurostimulation device (electrode assemblies) and a controller unit, where the controller unit can recharge the internal battery of the neurostimulation device (electrode assemblies) when not performing neurostimulation. When performing the stimulation to the users' ears, the controller unit can be used by the user to control stimulation intensity of the neurostimulation device (electrode assemblies). The stimulation data performed by the neurostimulation device (electrode assemblies) is collected by the controller unit and can be sent to a dedicated platform in the cloud. The controller unit and the connection to the internal application in the smartphone allows data transfer to an external cloud.

The controller unit communicates with the wireless neurostimulation device (electrode assemblies) for carrying out the ABVN stimulation. The controller unit allows the user to increase stimulation intensity of the wireless neurostimulation device (electrode assemblies) for carrying out the ABVN stimulation. The controller unit has a controller panel with a lighting display for ease of reference for the user to show the cumulated time of use, stimulation intensity, and the quality of skin contact to the ear.

Preferably, the neurostimulation device includes a photoethysmogram (PPG) or biosensor, or having at least one external input mechanism for inputting feedback data by the device user to the controller unit of the neurostimulation system, for estimating levels of hemoglobin and oxy hemoglobin within the user's circulation, where these data are used for calculating heart rate, HRV and breath rate and breathing phases of the user, or a parameter relating to the autonomic nervous system such as sympathovagal balance, or a parameter relating to movement activity or muscle response i.e. electromyography (EMG), of the user or a combination of the aforesaid parameters.

The PPG or biosensor, or external input mechanism for data feedback, is configured for detection of physiological markers such as low heart rate (bradycardia) and low breathing, in which case the stimulation will be stopped to avoid any health risks.

The neurostimulation system performing the ABVN stimulation treatment is configured with at least one wireless neurostimulation device (electrode assembly) for generating a stimulation pulse, with the neurostimulation system having at least one detection means that is configured to detect one or more parameter values, with the neurostimulation system having a controller unit that is suitable to set one or more parameters of the stimulation pulse delivered by the neurostimulation device (electrode assembly) based on detected parameter value or values.

The key advantage can thus be achieved with the stimulation provided being from a wireless neurostimulation device (electrode assemblies), and also the success of the treatment can take place in a manner specific to the user (personalized) or that the parameters that may influence stimulation, can determine a stimulation that increases the success of the treatment with respect to an unchangeable stimulation pattern i.e. stimulation protocol.

The neurostimulation system also includes a cloud platform that can integrate data obtained from devices or sensors, such as watches, bracelets or rings, among other external sensors, for continuous monitoring of heart rate activity, sleep cycle patterns etc. The analysis of these data allows, for example, to know the user's physiological states and define personalized stimulation treatments that can improve negative ailments.

According to a third aspect, the invention pertains a method for operating the auricular neurostimulation system, encompassing the following sequential steps:
a. Setup phase: the user inputs their personal information to create a profile (i.e. weight, age, medical history including symptom frequency and severity) and/or user's baseline physiology readings are obtained at rest with external sensors including metrics for heart rate, HRV, breathing rate, and autonomic system activity for sympathovagal balance.
b. Stimulation protocol: A stimulation protocol is designated to the user for unilateral, bilateral, synchronized or un-synchronized neurostimulation, that can be tailored to physiological, movement or muscle response (i.e. electromyography) trigger-activated neurostimulation.
c. Stimulation initiation: Upon placing the device onto the user's ears, the stimulation can be started.
d. Data collection: Throughout the stimulation, the biosensor or an external input device collects user physiological data and is transmitted to the controller unit.
e. Stimulation session completion and data storage: Once the stimulation is completed for the session, the controller unit stores the treatment session data.
f. Data transmission to the cloud: The controller unit transmits the treatment session data to the cloud platform to facilitate subsequent analyses.
g. Cloud data repository: The cloud-based platform stores the treatment session data received.
h. Algorithmic analyses: An algorithm analyses all the collected data, optimizing the stimulation parameters specific to individual users. Such optimized parameters are presented to the user and can be selected if preferred.

### BRIEF DESCRIPTIONS OF DRAWINGS

The characteristics, benefits and objects of the current invention will be evident to persons skilled in the art upon reading the subsequent detailed descriptions of illustrative embodiments of the invention, in conjunction with the accompanying drawings:

FIG. 1. Illustration of the external ear anatomy that can be targeted for auricular neurostimulation in a human.

FIG. 2. A schematic diagram showing effects of Vagus Nerve Stimulation (VNS). The Vagus Nerve (VN) activates ascending neural pathways leading to modulation of neural activity that can be stimulated via the Auricular Branch of Vagus Nerve (ABVN). The illustration shows the VN projects to the Nucleus Tractus Solitarii (NTS), and Dorsal Motor Nucleus (DMN), which further projects to higher brain regions including the locus coeruleus, hypothalamus, thalamus, basal ganglia, and forebrain cholinergic centers.

FIG. 3. Scheme with the components of the connected auricular neurostimulation system of the invention.

FIG. 4. A view of the neurostimulation device (electrode assembly) according to the invention, in which the inclination between the connecting portion is inclined relative to the vertical center line (y-axis) of the infinity holding portion.

FIG. 5. The neurostimulation device (electrode assembly) according to the invention, in which the holding portion and the connecting portion are manufactured from materials of similar hardness.

FIG. 6. The neurostimulation device (electrode assembly) according to the invention that shows the bud attached that contains the electrical components that generates the electrical pulses to carry out nerve stimulation.

FIG. 7. The neurostimulation device (electrode assembly) according to the invention on a side view.

FIG. 8. A view of the neurostimulation system's controller unit according to the invention.

FIG. 9. A view of the placement of the neurostimulation device (electrode assemblies) into the controller case, in which the holding portion is contained and connected to the controller unit through copper pogo pins that recharge the neurostimulation device (electrode assemblies).

FIG. 10. The neurostimulation device (electrode assemblies) stimulation for bilateral ear stimulation, in configurations for 10A. synchronized or 10B. unsynchronized neurostimulation of the ABVN.

FIG. 11. The user flow for setup of user account, device setup, data analysis and feedback of the neurostimulation system.

### DETAILED DESCRIPTION OF THE EXEMPLARY EMBODIMENTS

The object of the invention is an auricular neurostimulation device (electrode assembly) (1) wearable by a user that optimizes the stimulation of ABVN at the cymba conchae, as illustrated in FIG. 1.

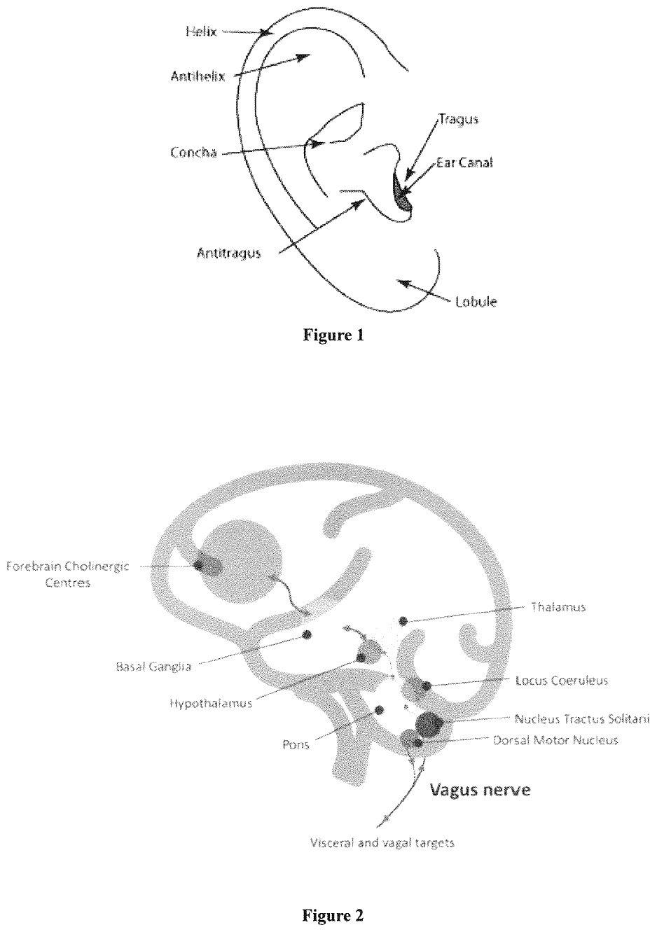

The neuroanatomy of ABVN stimulation is shown in FIG. 2. Neurostimulation at the ABVN via cymba conchae mediates stimulation of the Ramus Auricularis Nervi Vagi (RANV), activating the vagus nerve pathways to the NTS and DMN, and then activates the projections of higher brain regions that include the locus coeruleus, hypothalamus, thalamus, basal ganglia and forebrain.

The auricular neurostimulation device (electrode assembly) (1) of the invention is composed of the following components, as represented in FIG. 4, designed for placement into the user's ear:
At least 1 electrode in the cymba conchae: the device incorporates a minimum of one electrode (2) positioned in the cymba conchae (the only ear zone with 100% ABVN) and is configured as a working electrode.
Second electrode in the cymba conchae: a second electrode placed in the cymba conchae. In a preferred embodiment, both electrodes are configured as working electrodes for application of a voltage potential for optimizing the activation of the ABVN.
Biocompatible electrode assembly composition: electrodes are typically manufactured from biocompatible materials (including titanium, nickel titanium, platinum and platinum-iridium, non-toxic metals such as gold) and biocompatible polymers for adaptation to the user's ears for comfort, secure wear for providing accurate positioning and contact to the stimulation area. Further, electrode fitting does not block the ear canal i.e. does not impede auditory perceptions during use for carrying out ABVN stimulation.

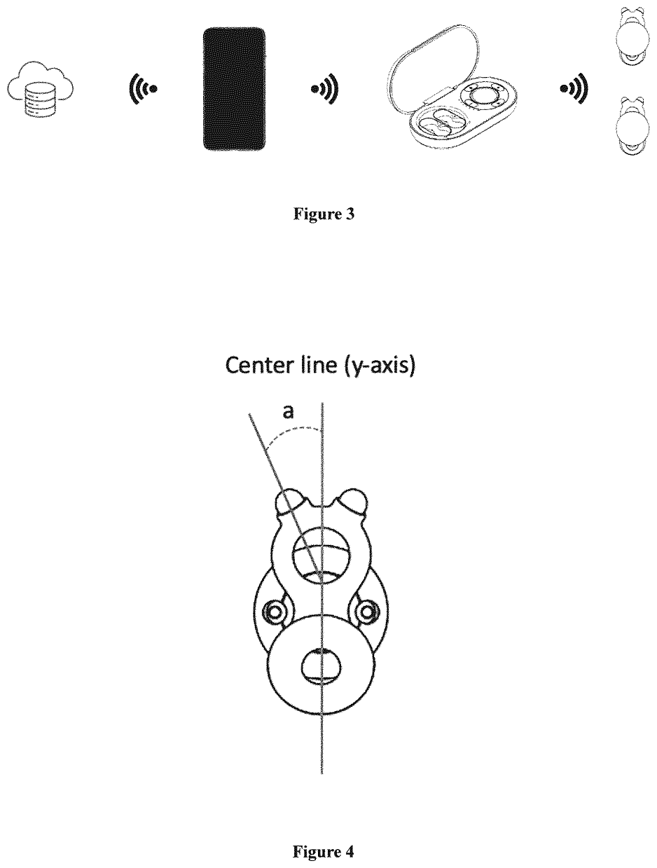

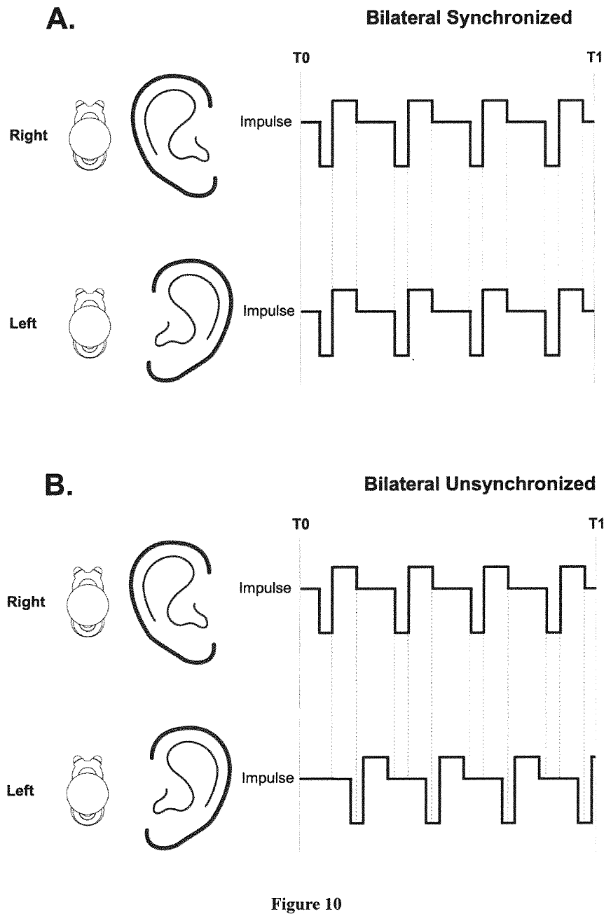

The auricular neurostimulation device (electrode assembly) (1) is integrated with electronic circuits enabling the following functionalities:
wireless ABVN Neurostimulation
synchronized or unsynchronized bilateral wireless ABVN neurostimulation (FIG. 10)
generation of stimulation patterns with variable duration, intensity, frequency of bursts and pulses, number of pulses per burst, pulse width, and pulse delays, among other factors
generation of stimulation patterns synchronized with the autonomic nervous system such as sympathovagal balance, and/or parameters relating to movement activity or muscle response i.e. electromyography (EMG), of the user.
wireless data transfer with the controller unit (FIG. 3)
detection of appropriate skin contact for sufficient neurostimulation conduction
the ergonomic design of neurostimulation device (electrode assembly) (1) protects the electronic circuits while ensuring ease, convenience, and stable placement into the users ear, or into the controller unit (12)

The auricular neurostimulation device (electrode assembly) (1) implements a range of stimulation protocols. Preferably, the stimulation protocols are based on waveform of rectangular, biphasic, symmetrical, and are synchronized for bilateral ABVN neurostimulation (FIG. 10A). This configuration is used to elicit the desired physiological effect of activations of nerve endings of ABVN fibres with higher efficiency.

Further, the stimulation protocols include burst stimulation for elevating endogenous responsiveness of ABVN nerve endings. Burst stimulation act to disrupt endogenous neural activities for heighted response that includes 1-20 bursts per second.

Further, stimulation protocols offer variations in parameters of pulse shape, pulse width, pulse frequency for user comfort and optimized stimulation. The stimulation intensity is adjustable between 0 and 3 mA. The pulse width determines engagement of specific ABVN fibres which includes Aβ Aδ and C (Safi et al., 2016). Short pulses waves of 10-300 μs are used to engage thick fibres of Aβ and avoid inductions of Aδ and C fibres, which transmit pain signals. The recruitment of desirable thick fibres are also dependent on frequency where 1-100 Hz are used.

Usability, stability and comfort-conventional auricular neurostimulators found in the state of the art often comprise of large generators connected via a cable to an accessory delivering an electrical voltage to varying ear regions. The size and weight of such setup limits the portability and practical use for users. In contrast, the auricular neurostimulation device (electrode assembly) (1) of this invention provides a compact, lightweight form-factor to ensure wearer comfort. The wireless auricular neurostimulation device (electrode assembly) (1) configuration and ear ergonomic design ensures the avoidance of ear canal obstruction or hinderance of auditory perceptions. The auricular neurostimulation device (electrode assembly) design allows for association with familiar products that facilitate user adoption and wireless data transfer further supports user convenience (FIG. 3). Furthermore, the ergonomic design of the auricular neurostimulation device (electrode assemblies) (1) ensures for stable placement into the users' ears that ensures good quality contact between electrodes (2) and the ABVN stimulation region.

The neurostimulation device (electrode assembly) (1) is an inventive electrode assembly for nerve stimulation, in particular for ABVN stimulation that includes at least one electrode (2) and an infinity shape (‘figure of eight’) holding portion (3) which is designed to hold the electrode assembly at the ear of the user.

As the activity of the nerve stimulation largely depends on a good contact between the electrode and the tissue to be stimulated, it has proven advantageous in practice when the connecting portion by means of which the electrode is connecting to the holding portion is inclined relative to the connecting portion (4).

The at least one electrode (2) preferably is connected to the holding portion of the infinity shape (‘figure of eight’) (3) via a connecting portion (4), wherein the connecting portion is inclined relative to the vertical center line (y-axis) of the infinity holding portion, at an angle of preferably about 10° to 40°, particularly preferably at about 30° (FIG. 4).

Due to the inclination of the connecting portion, and the infinity shape (‘figure of eight’) holding portion (3) are arranged with the access of the auditory canal of the user (5), a safe contact of the electrodes (2) with the skin of the user and a sufficient contact pressure are ensured even during movement of the user, for example while sleeping or during sport.

It has proven to be advantageous in practice when the holding portion (3) and the connecting portion (4) are manufactured from materials of similar hardness, in particular from materials with a Shore hardness in the range of about 60 Shore A to about 90 Shore A, particularly preferably in the range of about 70 Shore A to about 90 Shore A.

Due to the hardness of the material used, a good structural stability and rigidity of the electrode assembly can also be ensured.

It can be that the holding portion (3) is largely manufactured from a relatively soft material and the connecting portion (4) as well as a section of the holding portion adjoining the connecting portion can also be manufactured from materials of different hardness, wherein preferably the part manufactured from a relatively hard material is inserted, embedded, or sunk in the part manufactured from a relatively soft material. With the mixture/connection of harder materials and softer materials, the advantages of a good structure stability of the neurostimulation device (electrode assembly) (1) as a result of relatively hard materials can be combined with an increased wearing comfort due to relative soft materials.

Preferably, the part made of relatively hard material is inserted, embedded or sunk in the part made of relatively soft material or vice versa. This provides for generating a smooth surface, which increases the wearing comfort of the neurostimulation device (electrode assembly) (1) and also has hygienic advantages.

It is also conceivable to merely provide a framework of relatively hard material, which is completely or partly integrated or embedded in a portion made of soft material. For example, the holding portion can include such a framework which is sheathed with a softer material.

Furthermore, it has proven to be advantageous in practice when a cable associated with the at least one electrode (2) extends within the holding portion of the infinity shape ('figure of eight') (3).

Furthermore, it has proven to be advantageous when the neurostimulation device (electrode assembly) (1) includes two electrode contacts (2). Preferably, the electrode contacts (2) are made of coated titanium.

Another advantage of the present neurostimulation device (electrode assembly) (1) consists in that the at least one electrode (2) and the infinity shape (‘figure of eight’) holding portion (3) are arranged such that the access of the auditory canal of the users is not completely blocked and does not impede hearing when the neurostimulation device (electrode assembly) (1) is arranged in the ear of the user.

This is achieved in that the second ring, the lower part of the holding portion of the infinity shape (‘figure of eight’) (3) sits in the ear canal of the user. An additional full circle ring may be attached to the inner part of the lower portion (5), allowing further support for the neurostimulation device (electrode assembly) (1) to be secured into the ear of the user. In this way, the holding portion of the infinity shape (3) is tucked into the ear with sufficient contact pressure, and a safe contact of the electrode contacts (2) with the skin of the user is ensured. The neurostimulation device (electrode assembly) (1) supports good contact of electrode contacts (2) with the cymba conchae to maximize effects of stimulation. The advantage of the shape is that the lower portion of the infinity shape fits in the ear canal (5) but does not block or hinder the user's hearing to environmental sounds and therefore can be used for long durations and long wear times.

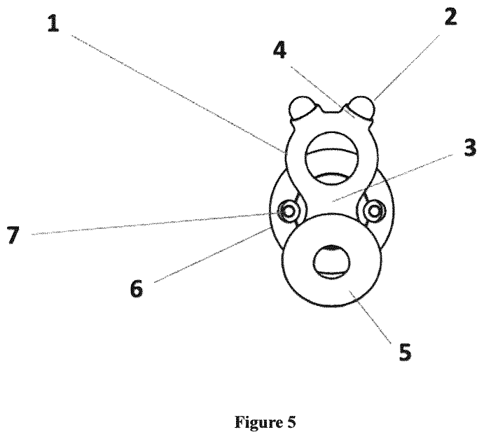

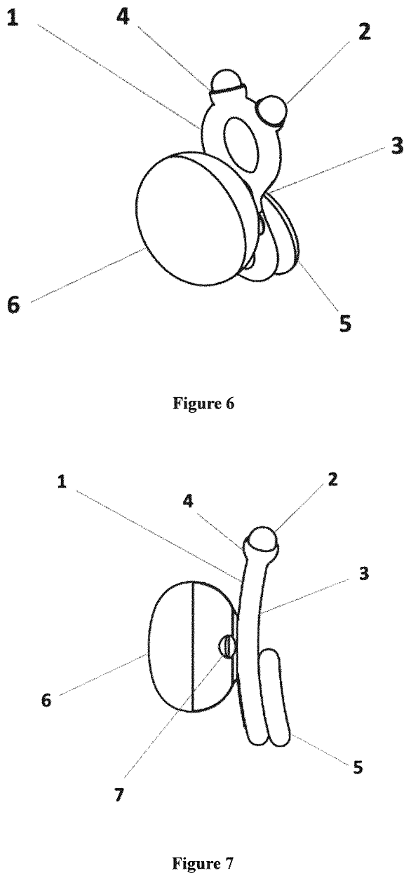

As shown in FIG. 5-7, the neurostimulation device (electrode assembly) (1) according to the invention in one embodiment includes two electrodes (2) and an infinity shape (‘figure of eight’) holding portion (3). The two electrodes are connected to the infinity shape (‘figure of eight’) holding portion via connecting portion (4).
The infinity shape (‘figure of eight’) holding portion (3) can include a full circle ring that can be attached to the inner part of the lower portion (5). The hollow center ensures it does not impede the auditory perceptions of the user to surrounding sounds of the environment.
The infinity shape (‘figure of eight’) holding portion (3) includes a bud (6) containing electrical components connected to the holding portion of the infinity shape (‘figure of eight’) (3) that generates the electrical pulses to carry out ABVN neurostimulation. There are two copper pogo pins (7) that allow for the recharging of the bud (6) containing the electrical components.
A cable associated with the electrode can be arranged in the holding portion (3).
The two electrodes (2) are positioned symmetrical to one another relative to the center-line (y-axis) of the holding portion of the infinity shape (‘figure of eight’) (FIG. 4).
As in the embodiment, a large part of the holding portion is made of relatively soft material, it may be advantageous to provide the necessary rigidity by means of a framework/inlay made of a relatively hard material inserted into the holding portion (4).

Efficiency and safety—the auricular neurostimulation device (1) of the invention targets the cymba conchae only, which is the only region of the ear where 100% nerve endings are from the ABVN. Among the auricular neurostimulation devices in this state of art, only Cerbomed stimulates the cymba at one ear (unilateral). In contrast, the present invention facilitates concurrent bilateral neurostimulation of the ABVN with either synchronized or unsynchronized stimulation (FIG. 10). Moreover, the present invention detects sufficient skin conduction to ensure quality contact for efficient neurostimulation.

According to a second object, the invention relates to an auricular neurostimulation system for ABVN stimulation comprising of the wireless neurostimulation device (electrode assemblies) (1) and a controller unit (8), where the controller unit can recharge the internal battery of the neurostimulation device (electrode assemblies) when not performing neurostimulation (12). When performing the stimulation to the users' ears, the controller unit (8) can be used by the user to control stimulation intensity (9) of the neurostimulation device (electrode assemblies) (1). The stimulation data performed by the neurostimulation device (electrode assemblies) (1) is collected by the controller unit (8) and can be sent to a dedicated platform in the cloud. The controller unit and the connection to the internal application in the smartphone allows data transfer to an external cloud (FIG. 3).

Preferably, the neurostimulation device includes a photoethysmogram (PPG) or biosensor, or having at least one external input mechanism for inputting feedback data by the device user to the controller unit (8) of the neurostimulation system, for estimating levels of hemoglobin and oxy hemoglobin within the user's circulation, where these data are used for calculating heart rate, HRV and breath rate and breathing phases of the user, or a parameter relating to the autonomic nervous system such as sympathovagal balance, or a parameter relating to movement activity or muscle response i.e. electromyography (EMG), of the user or a combination of the aforesaid parameters.

The PPG or biosensor, or external input mechanism for data feedback, is configured for detection of physiological markers such as low heart rate (bradycardia) and low breathing, in which case the stimulation will be stopped automatically to avoid any health risks.

The measurements taken by the sensor or from inputted data make it possible to provide adapted stimulation protocols that can achieve optimal vagal stimulation for the device user. This allows personalized stimulation treatments to the device user for reaching efficient ABVN neurostimulation that is much higher in effectiveness than other existing devices known in the art.

Further, the PPG or biosensor, or external input mechanism for data feedback, functions to detect parameters related to the autonomic nervous system such as sympathovagal balance, or a parameter relating to movement activity or muscle response i.e. electromyography (EMG), of the user. These inputs are harnessed to autonomously synchronize the neurostimulation device (1), to achieve elevate efficiency in activation of the vagus nerve.

Further, the wireless connection of the controller unit to a smartphone device through the mobile application and connection to an external cloud, establishes the complete neurostimulation system according to the present invention as illustrated in FIG. 3.

The auricular neurostimulation device (electrode assemblies) (1) are stored into the controller unit (8) that holds and recharges the neurostimulation device (electrode assemblies) (1) through pogo pins (7)(11) (FIG. 9). The controller unit (8) captures the data from the neurostimulation device (electrode assemblies) (1) and sends to a dedicated platform in the external cloud.

The neurostimulation system has a smartphone application that allows the user to engage with the auricular neurostimulation device (electrode assemblies) (1) and controller unit (8), for user reference and configurations of specific stimulation parameters. The mobile app establishes interchange of data with a dedicated platform in the cloud, receiving the data captured by the PPG or biosensor, and/or external input mechanism for data feedback, during stimulation sessions.

The neurostimulation system controller unit (8) of the invention is configured to detect parameters related to heart rate, HRV, and breath rate and breathing phases of the user, or a parameter relating to the autonomic nervous system such as sympathovagal balance, or a parameter relating to movement activity or muscle response i.e. electromyography (EMG), of the user or a combination of the aforesaid parameters. An adopted stimulation protocol can be assigned to the user for selection based on the user profile and feedback data received to achieve improved efficiency in activation of the user's vagus nerve.

The auricular neurostimulation device (electrode assemblies) (1) of the invention offers distinct advantages over known neurostimulation devices in the state of the art, with respects to usability, stability and comfort, efficiency, safety, and customization.

For safety, the PPG or biosensor, or external input mechanism for data feedback collected from the neurostimulation system of continuous monitoring of cardiac activity patterns through external wearables such as watches, bracelets and rings, is configured for detection of physiological markers such as low heart rate (bradycardia) and low breathing, in which case the neurostimulation will be stopped to prevent health risks.

Customization-the neurostimulation system of the invention includes therapeutic customization for selection of stimulation protocols personalized based on the user profile and feedback data received, that can be synchronized with the autonomic nervous system such as sympathovagal balance, and/or parameters relating to movement activity or muscle response i.e. electromyography (EMG), of the user. These customizations facilitate the user to achieve improved efficiency in activation of the user's vagus nerve.

The neurostimulation system performing the ABVN stimulation treatment is configured with at least one electrode (2) for generating a stimulation pulse and can be delivered wirelessly, with the neurostimulation system having at least one detection means that is configured to detect one or more parameter values, with the neurostimulation system having a controller unit (8) that is suitable to set one or more parameters of the stimulation pulse delivered by the electrode (2) based on detected parameter value or values.

In differing from fixed stimulation devices known from prior art, the stimulation protocols performed by the neurostimulation system can be modified/personalized to the user. With respect to one or more parameters such as the duration or strength of the pulse, it is rather dependent on one or more parameter values that are measured by external sensors.

The key advantage can thus be achieved with the stimulation provided being from a wireless auricular neurostimulation device (electrode assembly) (1), and also the success of the treatment can take place in a manner specific to the user (personalized) or that the parameters that may influence stimulation, can determine a stimulation that increases the success of the treatment with respect to an unchangeable stimulation pattern i.e. stimulation protocol.

The detection means are preferably configured in the form of one or more sensors.

The detection means can be configured to measure the parameter value or values such as the heart rate of the user in real time and the neurostimulation system controller unit (8) can be configured to set the stimulation pulse based on the parameter value or values measured in real time.

The detection means can be connected to a memory such that the parameter values measured by the detection means are stored in the memory and such that the neurostimulation system controller unit (8) is configured to set the stimulation pulse based on the parameter value or values stored in the memory. It would, for example, be conceivable that the data for physiological recordings such as heart rate and sleep cycle patterns are determined and stored over a longer period and that the stimulation pulse is delivered by the neurostimulation device (electrode assembly) (1) can be on the basis of stored values.

By way of example, the user parameters or parameters can be physiological signals of the user such as heart rate, breathing, sleep cycle patterns, that can be determined by means of an external sensor, a parameter for breathing, or a parameter relating to the autonomic nervous system such as sympathovagal balance, or a parameter relating to movement activity or muscle response i.e. electromyography (EMG), of the user or a combination of the aforesaid parameters.

It is conceivable, for example, that the neurostimulation system controller unit (8) is configured to control the wireless auricular neurostimulation device (electrode assembly) (1) such that the parameter value detected by means of the detection is adjusted to a desired value (9) or to a desired value range by means of the stimulation pulse.

By way of example, the neurostimulation system could be configured such that the ABVN stimulation influences the autonomic nervous system i.e. sympathovagal balance. If the vagal tone is directly detected i.e. through external sensors or by other means, the neurostimulation system controller unit (8) can be configured such that it specifically influences the stimulation of the neurostimulation device (electrode assembly) (1) and adapts the vagal tone to a desired value or desired value range.

The neurostimulation system controller unit (8) is configured to coordinate the stimulation pulse or pulses with periodically occurring physiological processes, in particular neuro-physiological processes of the user.

In accordance with a method of the neurostimulation system performing ABVN stimulation treatment that a stimulation pulse is generated by means of at least one wireless neurostimulation device (electrode assembly) (1), with one or more parameter values being detected in advance and with one or more parameters of the stimulation pulse delivered by the wireless neurostimulation device (electrode assembly) (1) being set by a neurostimulation system controller unit (8) based on the parameter value or values.

Unlike previous known devices for performing ABVN stimulation, the neurostimulation system in accordance with this invention for adaptive neurostimulation i.e. providing stimulation protocol that is not fixed or identical, but is based on one or more parameters specific to the user.

The present invention thus comprises of an evolved application from conventional ABVN neurostimulators, with various sensors that serve as input for control or variation of stimulation parameters.

A closed loop application is conceivable with regulation of a parameter to a desired value or in a desired value range.

The auricular neurostimulation device (electrode assemblies) (1) are controlled by wireless connectivity by the neurostimulator system controller unit (8). The controller unit can connect by wireless connectivity (e.g. Bluetooth) to external devices via a smartphone application, which in turn is connected to software in the cloud (FIG. 3). Through this connectivity the stimulation session data from the neurostimulation device electrode assemblies (1) can be transferred.

The auricular neurostimulation device (electrode assemblies) (1) are controlled by a neurostimulation system controller unit (8) for convenience and ease of use for the user. The neurostimulator system controller unit (8) has the size of a smartphone.

The neurostimulation system controller unit (8) communicates with the wireless neurostimulation device (electrode assemblies) (1) for carrying out the ABVN stimulation. The neurostimulation system controller unit (8) allows the user to increase stimulation intensity of the wireless neurostimulation device (electrode assemblies) (1) for carrying out the ABVN stimulation. The neurostimulation system controller unit has a controller panel (9) with lighting display for ease of reference for the user to show the cumulated time of use, the stimulation intensity, and the quality of skin contact to the ear.

When stimulation is not being performed, the neurostimulation device (electrode assemblies) are placed in an allocated position (10) in the neurostimulation system control unit (8) for recharging (12). The allocated position is constructed to contain the infinity shape (‘figure of eight’) neurostimulation device (electrode assemblies) (10).

When at least one neurostimulation device (electrode assembly) (1) is placed in the controller unit (8), there is recharge of the battery of the neurostimulation device (electrode assembly). There are two copper connector pogo pins that are allocated for the placement and connection for recharge (11) of the neurostimulation device (electrode assembly).

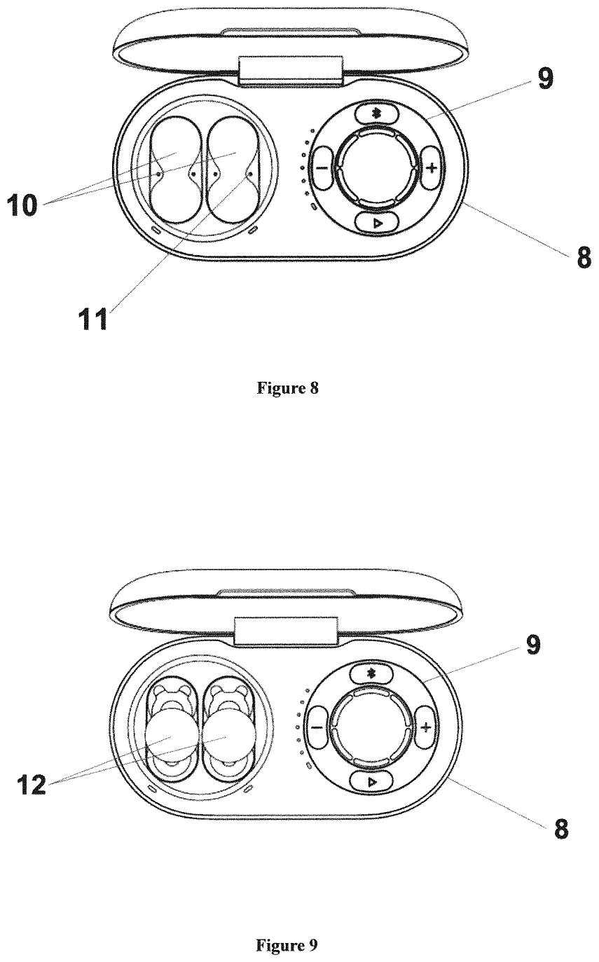

As shown in FIG. 8, a neurostimulation system controller unit (8) according to the invention that includes user panel for adjusting stimulation by the user (9). When stimulation is not being performed, the neurostimulation device (electrode assemblies) can be placed in the allocated position to hold the neurostimulation device (electrode assemblies) (12). The connection between the neurostimulation system controller unit (8) and the neurostimulation device (electrode assemblies) (1) are via two copper pogo pins that initiate the recharge (11), when the neurostimulation device (electrode assemblies) are placed into the neurostimulation system controller unit (12) as shown in FIG. 9.

The neurostimulation system controller unit (8) for ABVN stimulation is configured with at least one wireless neurostimulation device (electrode assembly) (1) that generates a stimulating pulse, wherein the neurostimulation system has at least one input device for inputting feedback data by the device user, wherein the neurostimulation system has a memory in which the feedback data are stored, and wherein the neurostimulation system can be adjusted to set one or more parameters of the stimulation pulse delivered by the wireless neurostimulation device (electrode assemblies) (1), that is dependent on user feedback or to propose the parameters of the stimulation pulse delivered by the neurostimulation device (electrode assemblies) (1) for a selection by the device user.

The electrical components of the neurostimulation system controller unit (8) are comprised of an electrical circuit that allows the following functionalities:
wireless control of ABVN neurostimulation
synchronized or unsynchronized bilateral wireless ABVN neurostimulation (FIG. 10)
generation of stimulation patterns with variable duration, intensity, frequency of bursts and pulses, number of pulses per burst, pulse width, and pulse delays, among other factors
generation of stimulation patterns synchronized with the autonomic nervous system such as sympathovagal balance, and/or parameters relating to movement activity or muscle response i.e. electromyography (EMG), of the user.
wireless data transfer with external devices (FIG. 3)
battery recharge of neurostimulation device (electrode assemblies)
detection of appropriate skin contact for sufficient neurostimulation conduction In accordance with the invention, the effect of the neurostimulation system is optimized by feedback of the users while taking account of defined parameters.

The neurostimulation system controller unit (8) for ABVN stimulation knows the defined parameters of the stimulation delivered by the wireless neurostimulation device (electrode assemblies) (1) and learns by the feedback of the user or users whether these parameters have been detrimental i.e. any negative effects on physiological state such as dizziness, pain, or whether the ailment has worsened. Based on the feedback, the neurostimulation system controller unit (8) can change the stimulation pulse of the wireless neurostimulation device (electrode assemblies) (1) for following treatments for improving the condition i.e. optimization based on feedback data.

The technical settings of the neurostimulation system controller unit (8) such as pulse duration, intensity, time of use, are set based on the feedback of the device user or users or proposed for setting by the user so that he/she can decide whether to activate this proposed setting.

It is conceivable that the feedback data stored in the memory of the neurostimulation system are individual to the user. This means that an optimization of the stimulation individual to the user is carried out.

It is also conceivable and covered by the invention that the feedback data stored in the memory of the neurostimulation system are not individual to the user, but rather relates to all device users for the particular disease or related condition.

The memory in which the feedback data are stored can be an integral component of the neurostimulation system or can be arranged as an external device component. This also applies in another respect to all the other components of the system.

The ‘neurostimulation system’ can be understood as a system of components that do not necessarily have to be arranged at one and the same location. The memory can thus, for example, be formed as a data pool by a server that is spatially separate from that part of the device the user actuates.

The neurostimulation system can have one or more external sensors by means of which one or more user parameters and/or external parameters can be detected, with the neurostimulation system controller unit (8) being configured also to set one or more parameters of the stimulation pulse that is delivered by the wireless neurostimulation device (electrode assemblies) (1) based on the user parameters and/or on the external parameters or to propose the parameters of the stimulation pulse delivered by the wireless neurostimulation device (electrode assemblies) (1) for selection by the user. In this case, these parameters such as heart rate parameters, sleep cycles, stress, physical activity, movement, muscle response etc., also enter into the parameter values of the stimulation pulse.

In accordance, a method for the neurostimulation system for performing ABVN stimulation treatment that can be with feedback data stored in a memory that are input by the device user by means of an input device and that are one or more parameters of the stimulation pulse delivered by the wireless neurostimulation device (electrode assemblies) (1) are set based on the feedback data or are proposed for selection to the user.

The collected data and the feedback data forms a data pool. This data pool has to be analyzed in particular for positive or negative correlations between the variables with respect to their effects on the perceived well-being or perceived change in negative state.

The conceivable applications of the method of the neurostimulation system in accordance with the invention are e.g.
Tinnitus
Addiction
Headache disorders
Bran fog
Confusion
Loss of concentration
Obesity
Cognitive impairment
Neuro-psychiatric behavioural disorders
Learning
Dementia
Stress
Physical performance
Inflammatory disorders
Autoimmune disorders
Persisting symptoms from COVID-19 infection
Immune system disorders
Eating disorders
Parkinson's disease
Alzheimer's diseases
High blood pressure
Diabetes
Stroke

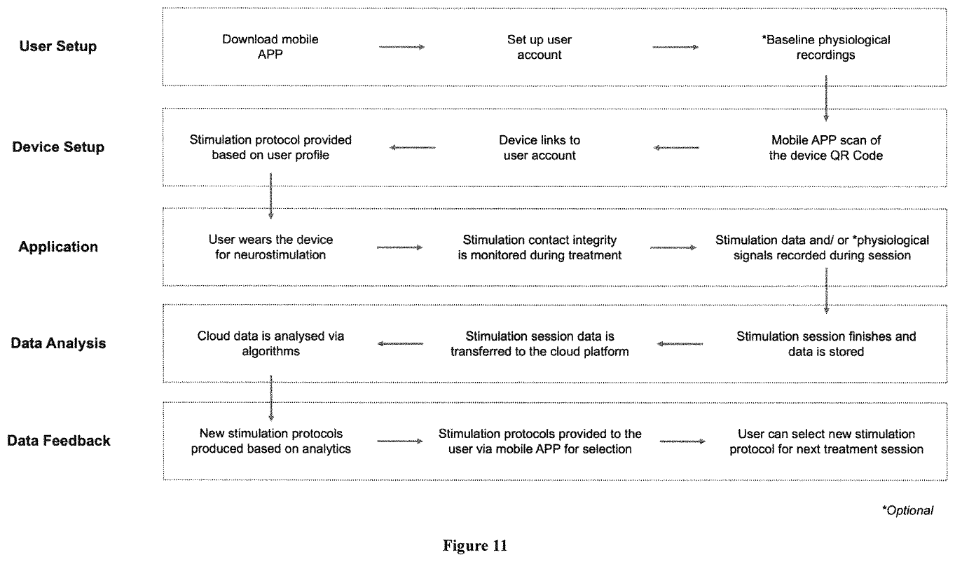

The operational method of the neurostimulation system in accordance with the present invention encompasses the following sequential steps (FIG. 11):

### A. Setup Phase

1. The user downloads the mobile application on their smartphone and creates an account on the system. For registration, the user is prompted to input accurate personal data including (i.e. weight, age, medical history including symptom frequency and severity). There is an optional step to take measurements of baseline physiology (metrics including heart rate, HRV, breathing rate, and autonomic system activity for sympathovagal balance) using commonly worn external wearable sensors such as watches, bracelets or rings, among other external sensors.

2. A suitable stimulation protocol is assigned depending on the user's profile based on data groups and statistical studies. As the auricular neurostimulation device is adopted, the analysis of the data captured by PPG or biosensor, or external input mechanism for data feedback, allows these stimulation protocol to be customized.

3. Upon successful account registration, the user proceeds to log into the application to establish a connection with the auricular neurostimulation device. Through the mobile application, the user is prompted to scan the QR code that is unique to the auricular neurostimulation device. This connection facilitates the assignment of the device's serial number with the user account.

### B. Applying Abvn Neurostimulation

1. The user selects a stimulation protocol from those available depending on his/her needs (unilateral, bilateral, synchronized or un-synchronized, and/or physiological or movement or muscle response i.e. electromyography (EMG) triggered). The mobile app sends the data of the protocol selected to the neurostimulation system controller unit (8).

2. Upon user placement of the wireless neurostimulation device (electrode assemblies) (1) following removal from the neurostimulation system controller unit (8), the controller unit will automatically detect for sufficient skin contact via the neurostimulation device (electrode assemblies) (1) integrated sensor. If the neurostimulation device (electrode assemblies) (1) are well placed in the user's ear and the impedance of electrode contacts (2) with the ABVN area is good, the neurostimulation system will allow the user to start stimulation using the neurostimulation system controller unit (8).

3. The neurostimulation system controller unit monitors the stimulation intensity and duration of use (9). When maximum stimulation time elapses, or if electrode skin contact is compromised, or when the user takes off the neurostimulation device (electrode assemblies) (1) and re-inserts to the neurostimulation system controller case (12), the stimulation stops automatically.

4. During stimulation, the PPG or biosensor data, or data collected from external input devices, of the user's physiological metrics is transmitted to the neurostimulation system via Bluetooth.

5. Following stimulation completion, the neurostimulation system controller unit (8) stores the session data (including date, time, stimulation duration, stimulation parameters and user's physiological metrics). The batteries of the neurostimulation device (electrode assemblies) (1) are recharged with return to the neurostimulation system controller unit (8).

6. The neurostimulation system controller unit (8) transmits the data of each stimulation session to the mobile application. From here, the mobile application sends the data to the cloud for analyses.

7. the Cloud-based Platform Stores the User Data Sessions.

8. An algorithm systematically processes all user-specific data. If a more suitable stimulation protocol is available based on the user's profile, the platform sends the values to the application, so that the optimized stimulation protocol can be selected if the user wishes.

### REFERENCES

Cai et a.l., Neurosci Lett 2019; 703:104-110

Baig et al., J Stroke Cerebrovasc Dis 2019; 28(12): 104348

Bauer et al., Brain Stim 2016; 9(3): 356-363

Badran et al., Brain Stim 2018; 11(3): 492-500

Frangos et al., Brain Stim 2015; 8(3): 624-36

Huang et al., BMC Complement Altern Med 2014; 14:203

Jacobs et al., Neurobiol Aging 2015; 36(5): 1860-7

Kaczmarczyk et al., J Neurochem 2017; jnc. 14284

Ko, Auton Neurosci 2021; 235:102858

Peuker and Filler, Clin Anat 2002; 15(1) 35-7

Redgrave et al., J Stroke Cerebrovasc Dis 2018; 27(7)1998-2005

Rong et al., Clin Sci (Lond) 2014

Safi et al., Anat Rec 2016; 299(9) 1184-91

Stefan et al., Epilepsia 2012; 53(7): e115-8

Straube et al., J Headache Pain 2015; 16:543

Ventureyra, Childs Nerv Syst 2000; 16(2): 101-2

### Patents

[1] EP 3100764(A1 ); 07-12-2016

[2] DE 102015007215(B3 ); 25-02-2016

While the present invention described in reference to preferred embodiments, individuals whom possess ordinary expertise in the field can effectuate numerous modifications and adjustments without deviating from the scope of this invention, which is defined by the appended claims.

1. An auricular neurostimulation device (electrode assembly) (1) that is wireless and wearable by a user and is configured to provide concurrent bilateral neurostimulation of the Auricular Branch of Vagus Nerve (ABVN) of the user's ears, wherein,
the auricular neurostimulation device (1) is wireless comprising of at least one electrode contact (2) positioned to the cymba conchae for administering electrical stimulation exclusively to the nerve ramifications of the ABVN;
characterized in that
the auricular neurostimulation device (1) is structured in the form of a wireless earbud, integrating a compact design (6) that avoids full obstruction of the user's ear canals (5) or avoids inhibition of the user's auditory perceptions wherein,
the at least one electrode contact (2) is positioned on the neurostimulation device (1) ear-mold that encompasses the ABVN anatomical region,
a conduction sensor, designed within the ear-mold, and
a compacted faceplate design (6) encapsulated with all electrical components on a Printed Circuit Board (PCB) connected to the holding portion of an infinity shape (‘figure of eight’) (3), that generates the electrical pulses to carry out ABVN stimulation.

2. The auricular neurostimulation device (electrode assembly) (1) according to claim 1 where in the at least one electrode contact (2) is made of biocompatible materials and biocompatible polymers for adaptation to user's ears for comfort, secure wear providing accurate positioning and contact to the stimulation areas.

3. The auricular neurostimulation device (electrode assembly) (1) according to claim 1 for ABVN stimulation, comprising at least one electrode contact (2) and an infinity-shaped (‘figure of eight’) holding portion (3) designed to hold the neurostimulation device (electrode assembly) (1) at the ear of the user, wherein the at least one electrode contact (2) is connected to the holding portion of the infinity shape (‘figure of eight’) (3) via a connecting portion (4), and wherein the connecting portion is inclined relative to the vertical center line (y-axis) of the infinity holding portion (3) at an angle of about 10° to 40°.

4. (canceled)

5. The auricular neurostimulation device (electrode assembly) according to claim 3, characterized in that the holding portion (3) and the connecting portion (4) are manufactured from materials of similar hardness with a Shore hardness in the range of about 60 Shore A to about 90 Shore A, and wherein the holding portion (3) is largely manufactured from a relatively soft material, and the connecting portion (4) as well as a section of the holding portion adjoining the connecting portion can be manufactured from materials of different hardness, with the relatively hard material preferably being inserted, embedded, or sunk into the relatively soft material.

6. (canceled)

7. The auricular neurostimulation device (electrode assembly) (1) according to claim 1, characterized in that a cable associated with at least one electrode contact (2) extends along or within the holding portion of the infinity shape (‘figure of eight’) (3), and that the at least one electrode contact (2) and the infinity shape (‘figure of eight’) holding portion (3) are arranged such that the access to the auditory canal of the user is not completely blocked and does not impede the auditory perception of the user when the neurostimulation device (electrode assembly) (1) is arranged in the ear.

8. (canceled)

9. The auricular neurostimulation device (electrode assembly) (1) according to claim 1, wherein a lower portion of the holding portion of the infinity shape (‘figure of eight’) forms a ring, and an additional full circle ring may be attached to the inner part of the lower portion (5), allowing further support for the neurostimulation device (electrode assembly) (1) to be secured into the ear of the user.

10. The auricular neurostimulation device (electrode assembly) (1) according to claim 1, wherein the electronic circuit comprises of:
a central circuit for generation of stimulation patterns;
a voltage amplifier that can modulate the potential difference supplied by a battery;
a charger circuit;
a circuit for detection of impedance;
a battery that is rechargeable.

11. An auricular neurostimulation system comprising: a bilateral wireless neurostimulation device (1) and a controller unit (8), wherein the controller unit recharges an internal battery of the neurostimulation device when placed into the controller unit and where the stimulation data performed by the neurostimulation device (1) is collected by the controller unit (8) and is sent to a dedicated cloud-based platform, and wherein the auricular neurostimulation system further includes at least one external input mechanism for inputting feedback data by the device user to the controller unit (8), for estimating levels of hemoglobin and oxyhemoglobin within the user's circulation, and using this data to calculate heart rate, heart rate variability (HRV), breath rate, breathing phases, autonomic nervous system parameters such as sympathovagal balance, or movement activity or muscle response (i.e., electromyography (EMG)), or a combination thereof.

12. (canceled)

13. The auricular neurostimulation system according to claim 11, wherein the controller unit (8) has a PCB electronic circuit configured to do at one or more of the following:
perform wireless control of ABVN neurostimulation;
generate synchronized or unsynchronized bilateral wireless ABVN neurostimulation;
generate stimulation patterns with variable duration, intensity, frequency of bursts and pulses, number of pulses per burst, pulse width, and pulse delays,
generate of stimulation patterns synchronized with the autonomic nervous system such as sympathovagal balance, and/or parameters relating to movement activity or muscle response i.e. electromyography (EMG), of the user;
conduct wireless data transfer with external devices;
conduct battery recharge of neurostimulation devices and
detect appropriate skin contact for sufficient neurostimulation conduction.

14. The auricular neurostimulation system according to claim 11, characterized in that it incorporates a multitude of stimulation protocols tailored for bilateral ABVN neurostimulation, where the intensity of the electrical current, pulse width, and pulse frequency is variable; and the stimulation protocols are based on rectangular, bi-phasic symmetrical waveforms, wherein the stimulation protocols applied through the neurostimulation device (1) are personalized for each user according to therapeutic requirements, and wherein an electrical voltage difference applied any electrodes is measured in real-time to provide metrics of impedance of the contact electrodes to the skin.

15. (canceled)

16. (canceled)

17. The auricular neurostimulation system comprising a neurostimulation device (1) according to claim 11 further comprises: a smartphone having a smartphone application; where the controller unit (8) and a connection to the smartphone application in the smartphone allows data transfer to an external cloud; and where the user can use the smartphone application to interact with the neurostimulation device.

18. The auricular neurostimulation system according to claim 11, having at least one input device for inputting feedback data by the device user, characterized in that the neurostimulation system has memory in which the feedback data are stored, and in that the neurostimulation system has a controller unit (8) that is suitable to set one or more parameters of a stimulation pulse delivered by the neurostimulation device (1) based on the feedback data or to propose parameters of stimulation (a stimulation protocol) delivered by the neurostimulation device (1) for selection by the device user, wherein the feedback data stored in the memory are either individual to the user or not individual to the user, but rather to all the neurostimulation system users for a specific/or related disease of a majority of users.

19. (canceled)

20. (canceled)

21. The auricular neurostimulation system according to claim 18, characterized in that the memory is either an integral component of the neurostimulation system or is arranged as an external device component, with at least a 2-point communication link from the input controller unit (8) to the memory, wherein the neurostimulation device (1) is wireless, and the neurostimulation system controller unit (8) is portable and has a size similar to that of a smartphone.

22. (canceled)

23. (canceled)

24. The auricular neurostimulation system according to claim 11, characterized in that the neurostimulation system has one or more sensors by means of which one or more user parameters and/or external parameters can be detected, and in that the controller unit (8) is configured also to set one or more parameters of a stimulation pulse or pulses delivered by the wireless neurostimulation device (1) based on the user parameters and/or on the external parameters or to propose the parameters of the stimulation pulse delivered by the electrode for selection by the device user.

25. The auricular neurostimulation system according to claim 11, characterized in that the neurostimulation device (1) having at least one electrode (2) for generating a stimulation pulse wirelessly, with the neurostimulation system having at least one detection means that is configured to detect one or more parameter values, with the neurostimulation system having a controller unit (8) that is suitable to set one or more parameters of the stimulation pulse delivered by the wireless neurostimulation device (1) based on the detected parameter value or values.

26. (canceled)

27. The auricular neurostimulation system according to claim 25, characterized in that the parameter or parameters whose values are measured by the at least one detection means are user parameters or external parameters, wherein the user parameter is the physiological signals of the user such as heart rate, that can be determined by means of an ECG sensor, a parameter relating to breathing, or a parameter relating to the autonomic nervous system such as sympathovagal balance, or a parameter relating to movement activity or muscle response i.e. electromyography (EMG), of the user or a combination of any of the aforesaid parameters.

28. (canceled)

29. The auricular neurostimulation system according to claim 25, characterized in that the controller unit (8) is configured to control the neurostimulation device (1) wirelessly such that the parameter value detected by means of the at least one detection means is adjusted to a desired value or to a desired value range by means of the stimulation pulse.

30. The auricular neurostimulation system according to claim 24, characterized in that the controller unit (8) is configured to coordinate the stimulation pulse or pulses with periodically occurring physiological processes, in particular neuro-physiological processes of the user.

31. The methods for operating the auricular neurostimulation system of claim 18 comprising the following steps of:
a. Prior to stimulation:
creating a user account on the system via a smartphone application with the user entering personal data;
assigning a suitable stimulation protocol depending on the user's profile based on data groups and statistical studies;
having the user log into the mobile application to establish a connection with the auricular neurostimulation device through scanning the device QR code, which facilitates the assignment of the device's serial number with the user account;
b. After completion of stimulation:
storing any session data (including date, time, stimulation duration, stimulation parameters and user's physiological metrics) in the auricular neurostimulation system controller unit (8);
recharging any batteries of the auricular neurostimulation (1) with return to the neurostimulation system controller unit;
transmitting the data of each stimulation session from the neurostimulation system controller unit (8) to the mobile application, wherein the data is then sent to the clou; for analyses;
storing the user data sessions in a cloud based platform;
wherein an algorithm systematically processes all user-specific data such that a more suitable or adopted stimulation protocol may be provided based on the user's profile, and wherein the platform will send the values to the application, so that the optimized or personalized stimulation protocol can be selected.
